(19) United States
(12) Patent Application Publication
(10) Pub. No.: US 2022/0288286 A1
(43) Pub. Date: Sep. 15, 2022

(54) ANTITHROMBOTIC COATING COMPOSITION AND ANTITHROMBOTIC COATING METHOD USING SAME

(71) Applicant: NOANIX CORPORATION, Cheongjusi (KR)

(72) Inventor: Hyungjoon CHOI, Cheongjusi (KR)

(73) Assignee: NOANIX CORPORATION, Cheongjusi (KR)

(21) Appl. No.: 17/384,145

(22) Filed: Jul. 23, 2021

(30) Foreign Application Priority Data
Mar. 9, 2021 (KR) 10-2021-0030542

(51) Int. Cl. A61L 33/06; A61L 29/08; A61L 33/00; A61L 29/16; A61K 31/727; A61K 31/685; A61K 31/737

(52) U.S. Cl. CPC A61L 33/062; A61L 29/085; A61L 33/0011; A61L 33/0041; A61L 29/16; A61L 33/0094; A61K 31/727; A61K 31/685; A61K 31/737; A61L 2300/42; A61L 2420/02; A61L 2420/08; A61L 2300/236; A61L 2300/232; A61L 2300/216; A61L 2420/06

(57) ABSTRACT

Disclosed is an antithrombotic coating composition and a coating method using the same. The composition is highly hydrophilic and biocompatible, thereby enabling a thin and flexible coating layer. The composition is suitably used for coating vascular catheters, stents, guide wires, and other invasive medical devices.

### CROSS REFERENCE TO RELATED APPLICATION

The present application claims priority to Korean Patent Application No. 10-2021-0030542, filed Mar. 9, 2021, the entire contents of which is incorporated herein for all purposes by this reference.

### BACKGROUND OF THE DISCLOSURE

### 1. Field of the Disclosure

The present invention relates to a composition for an antithrombotic coating and an antithrombotic coating method using the same.

### 2. Description of the Related Art

Recently, the development of medical devices that complement surgical procedures has been actively carried out. For example, such medical devices include various vascular catheters for circulatory system treatment such as aortic surgery and stents that reinforce the arterial wall and prevent occlusion after angioplasty. Heart valves, pacemakers, and orthopedic implants also belong to the extended list of such medical devices.

The implantation of a stent using balloon dilatation into an occluded vessel has been performed for the past several years. Although stents reduce the risk of occlusion of vessels, they do not yet completely prevent restenosis. The inventors of the present invention have made efforts to prepare a novel antithrombotic coating solution to prevent restenosis.

### CITATION LIST

### Patent Literature

Korean Patent Application Publication No. 10-2015-0119178

### SUMMARY OF THE DISCLOSURE

The present invention has been made in view of the problems occurring in the related art, and an objective of the present invention is to provide an antithrombotic coating composition containing silver nitrate and an antithrombotic coating method using the same.

In order to achieve the above objective, the present invention provides an antithrombotic coating composition including: a first coating solution including at least one selected from the group consisting of an acrylic compound and a polyurethane compound; a second coating solution including a polysaccharide and an antithrombotic substance; and a cross-linking agent.

In one embodiment of the present invention, the antithrombotic substance may be at least one selected from the group consisting of heparin, hirudin, H-heparin, HSI-heparin, streptokinase, neurokinase, fucoidan, and 2-methacryloyloxyethyl phosphorylcholine (MPC).

In one embodiment of the present invention, the first coating solution may be bound to a substrate, and the second coating solution may be bound, by the cross-linking agent, to the first coating solution bound to the substrate.

In one embodiment of the present invention, the acrylic compound may be an acrylate polymer.

In one embodiment of the present invention, the polyurethane compound may be polyether polyurethane.

In one embodiment of the present invention, the polysaccharide may be hyaluronic acid.

In one embodiment of the present invention, the cross-linking agent may be a polyaziridine-based or polyisocyanate-based cross-linking agent.

In one embodiment of the present invention, the second coating solution may further include polyether polyurethane.

In one embodiment of the present invention, the polysaccharide and the polyether polyurethane may be present in a weight ratio of 10:0.5 to 2.

Another objective of the present invention is to provide an antithrombotic coating method including: a) coating a surface of a substrate with a first coating solution containing at least one selected from the group consisting of an acrylic compound and a polyurethane compound; b) drying the coated substrate resulting from step a); c) coating the dried substrate resulting from step b) with a second coating solution containing a polysaccharide and an antithrombotic substance; and d) drying the coated substrate resulting from step c).

A further objective of the present invention is to provide an antithrombotic coated substrate prepared by the coating method.

The antithrombotic coating composition of the present invention is highly hydrophilic and biocompatible. Therefore, the antithrombotic coating composition of the present invention has the advantages of enabling a thin and flexible coating layer and exhibiting an antithrombotic function. The antithrombotic coating composition of the present invention is suitable for coating vascular catheters, stents, guide wires, and other invasive medical devices.

### BRIEF DESCRIPTION OF THE DRAWINGS

The above and other objectives, features, and other advantages of the present invention will be more clearly understood from the following detailed description taken in conjunction with the accompanying drawings, in which:

FIG. 1 is a view illustrating comparison in the frictional force of a sample surface between the presence and absence of an additive in a second coating solution;

FIG. 2 is a view illustrating comparison in friction force between a sample surface coated with an antithrombotic coating solution of the present invention and a sample surface which is not coated;

FIG. 3 is a view illustrating the platelet adhesion test result of a control group (Co—Cr alloy disk);

FIG. 4 is a view illustrating the platelet adhesion test result of a heparin-coated substrate;

FIG. 5 is a view illustrating the platelet adhesion test result of a fucoidan-coated substrate;

FIG. 6 is a view illustrating the platelet adhesion test result of an MPC-coated substrate; and

FIG. 7 is a view showing photographs of tubes to illustrate the visual observation results of the antithrombotic effect, in which the photographs from the leftmost respectively show a clean tube that is not yet used for testing, a tube used as a control group, a tube used as a heparin-coated substrate, a tube used as a fucoidan-coated substrate, and a tube used as an MPC-coated substrate.

### DESCRIPTION OF THE PREFERRED EMBODIMENTS

The present invention provides an antithrombotic coating composition including: a first coating solution including at least one selected from the group consisting of an acrylic compound and a polyurethane compound; a second coating solution including a polysaccharide and an antithrombotic substance; and a cross-linking agent.

In the present invention, the antithrombotic substance is at least one selected from the group consisting of heparin, hirudin, H-heparin, HSI-heparin, streptokinase, neurokinase, fucoidan, and 2-methacryloyloxyethyl phosphorylcholine (MPC).

In the present invention, the polyurethane is a generic term for polymer compounds with a urethane bond, which are obtained by combining an alcohol group and an isocyanic acid group. In the present invention, any polyurethane compound can be used if exhibits the characteristics of polyurethane. Various polyurethane compounds that can be derived from compounds having an alcohol group or an isocyanic acid group, within a range of modifications that can be easily achieved by those skilled in the art, may be used as the polyurethane compound in the present invention.

In the present invention, the term “acrylic compound” refers to a resin (polymer) in which the monomer of acrylic acid, acrylate, methacrylic acid, or any derivative thereof is repeatedly present. The acrylic compound may be a homopolymer having the same monomers therein or a copolymer having two or more different monomers therein.

In the present invention, the term “polysaccharide” refers to a polymeric carbohydrate molecule in which monosaccharide units are linked by glycosidic bonds to form a long chain.

When a material is expressed as being hydrophilic, it means that liquid droplets do not readily form beads on the surface of the material, come into contact with the surface of the material with a contact angle of 45°, and tend to easily spread on the surface the material.

The antithrombotic coating composition of the present invention may include one or more additives commonly used in coating formulations, for example, surfactants, preservatives, viscosity modifiers, pigments, dyes, and other additives known to those skilled in the art.

In the present invention, the first coating solution may be bound to a substrate, and the second coating solution may be bound, by the cross-linking agent, to the first coating solution bound to the substrate.

The second coating solution that is a hydrophilic solution among the components of the coating composition of the present invention imparts a coated medical device with lubricity and antithrombotic activity when the coated medical device comes into contact with an aqueous medium. The first coating solution may form an intermediate coating layer (hereinafter, also referred to as first coating layer) between a hydrophilic coating layer (hereinafter, also referred to as second coating layer) formed from the second coating solution and the surface of a medical device and have excellent adhesion to the surface of the medical device substrate, when the medical device is coated with the coating composition of the present invention.

The cross-linking agent serves to link the polymer in the second coating solution to the polymer in the first coating solution. Accordingly, the hydrophilic polymer in the second coating solution is chemically linked to the polymer in the intermediate coating layer formed from the first coating solution. Since the first coating solution that is cured absorbs a small amount of water, the adhesion of the coating composition to the surface of the medical device can be maintained even when the coated medical device comes into contact with an aqueous medium. In addition, the second coating layer bound to the first coating layer may provide lubricity.

In the present invention, any compound can be used as the cross-linking agent without limitation if it can bind the first coating layer formed from the first coating solution to the second coating layer formed from the second coating solution. Preferably, it may be an aziridine-based cross-linking agent or an isocyanate-based cross-linking agent but is not limited thereto.

In the present invention, the term “substrate” refers to a target to which the antithrombotic coating composition of the present invention is applied. Preferably, the substrate may be an invasive medical device. For example, it may be one of conventional medical devices including catheters, balloon catheters, guide wires, endotracheal tubes, implants, and the like. However, in the present invention, the substrate is not limited thereto.

In the present invention, the coating composition may be biocompatible.

When a substance is expressed as being biocompatible, it means that the substance does not cause any harm or side effects to the living body when it is administered or applied to the living body.

In the present invention, the polyurethane may be polyether polyurethane but may not be limited thereto.

In the present invention, the acrylic compound may be an acrylate polymer. Examples of the monomer of the acrylate polymer include methyl acrylate, ethyl acrylate, butyl acrylate, cyclohexyl acrylate, hexyl acrylate, 2-ethylhexyl acrylate, stearyl acrylate, and the like but are not limited thereto.

In the present invention, the polysaccharide may be hyaluronic acid but may not be limited thereto.

In the present invention, the cross-linking agent may be a polyaziridine-based or polyisocyanate-based cross-linking agent but may not be limited thereto.

In the present invention, the second coating solution may further, but not limitedly, include polyether polyurethane.

In the present invention, the weight ratio of the polysaccharide and the polyether polyurethane is 10:0.5 to 2, preferably 10:0.5 to 1.5, and most preferably 10:1. In addition, the coating solution having such a ratio may provide a coated surface that is less frictional and durable.

In another aspect of the present invention, there is provided antithrombotic coating method including a) coating a surface of a substrate with a first coating solution containing at least one selected from the group consisting of an acrylic compound and a polyurethane compound; b) drying the coated substrate resulting from step a); c) coating the dried substrate resulting from step b) with a second coating solution containing a polysaccharide and an antithrombotic substance; and d) drying the coated substrate resulting from step c).

The coating may be performed using a coating method that is commonly used in the art using an antithrombotic coating solution. Preferably, dip coating may be used as the coating method, but the coating method is not limited thereto.

In a further aspect of the present invention, there is provided an antithrombotic coated substrate prepared by the antithrombotic coating method. The coating composition of the present invention is highly hydrophilic and biocompatible. Therefore, the coating composition of the present invention has the advantages of providing a thin and flexible coating layer and exhibiting an antithrombotic function. The coating composition of the present invention is suitable for coating vascular catheters, stents, guide wires, and other invasive medical devices.

Hereinbelow, examples will be described to aid in understanding the present invention. However, the examples described below are provided only to facilitate the understanding of the present invention and thus the details in the examples should not be construed to limit the scope of the present invention.

### Example 1: Preparation of Coating Solution

Preparation of First Coating Solution

10 g of 1085 A 15 resin, which is a polyether polyurethane compound, was weighed and put into a vessel, and 1 L of a liquid mixture containing ethanol and water in a ratio of 9:1 was added thereto. Then, the resulting mixture was stirred at 600 to 800 rpm using a magnetic bar so that a solid phase can be completely dissolved. Then, HD-100, which is a polyaziridine-based cross-linking agent, was quantitatively added (3 g relative to 1 L) with a dropping pipette, and the resulting mixture containing HD-100 was stirred at 400 to 400 rpm at room temperature for 5 minutes using a magnetic bar to obtain a first coating solution.

Preparation of Second Coating Solution

50 g of hyaluronic acid powder was quantified in a 1 L bottle, and then 500 ml of ethanol was poured into the bottle and stirred to disperse the hyaluronic acid powder well.

Then, 500 ml of distilled water (DW) was slowly poured while stirring until the hyaluronic acid powder was completely dissolved. After confirming that the hyaluronic acid powder was completely dissolved, the mixture was stirred until bubbles disappeared. Then, 5 g of a polyether polyurethane-based compound, 5604A, was added and stirred until the compound was dissolved to prepare a second coating solution. Then, 0.5 g of an antithrombotic substance which is one selected from among heparin, fucoidan, and MPC was added and stirred to complete the preparation of an antithrombotic coating composition according to the present invention.

### Example 2: Process of Coating Substrate with Antithrombotic Coating Composition

First, an object to be coated (hereinafter, referred to as a coating target) was washed with a solution of isopropyl alcohol (IPA) and distilled water and then dried. Next, the coating target was immersion in the first coating solution prepared by the method of Example 1 so as to be primarily coated and then dried in an oven at 60° C. for 10 minutes. Next, the target was cooled at room temperature for about 5 to 10 minutes, then immersed in the second coating solution prepared by the method of Example 1 so as to be secondarily coated and dried in an oven at 60° C. for 120 minutes.

### <Example 3: Observation of Increase in Lubricity of Second Coating Solution Containing Additive

For a case where polyether polyurethane as an additive was included in the second coating solution, a frictional force test was performed to check for the improvement in the property of the coating solution. The test was performed in a manner that an upper portion of a catheter sample coated by the method of Example 2 was held with fixing tongs installed above a water tank in a test apparatus. Then, the catheter sample was pulled at a constant speed, and the frictional force was measured with a friction force sensor. In this way, the smoothness of the surface of the catheter sample was checked 10 times.

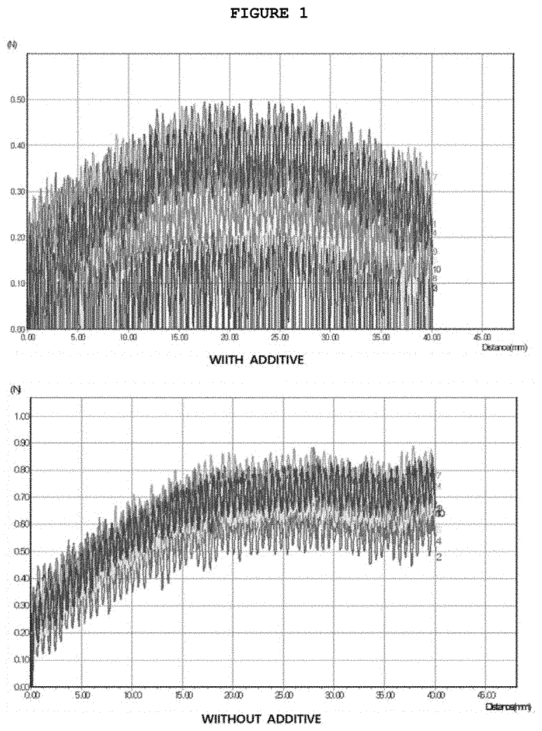

As a result, as illustrated in FIG. 1, the frictional force measured from the catheter sample coated with the second coating solution containing an additive had a remarkably low value. The test results prove that when polyether polyurethane is included as an additive in the second coating solution, the surface smoothness of a substrate is significantly increased due to the synergistic effect with the hyaluronic acid.

### Example 4: Comparison of Frictional Force of Substrate Before and After Coating

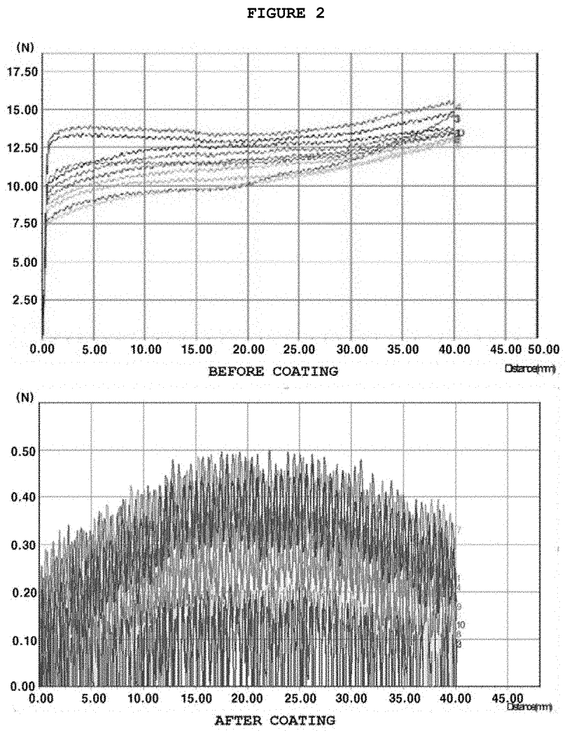

In order to check the effect of the coating, a frictional force test was performed on a sample surface before and after the sample surface was coated as in the method of Example 3. As a result, as shown in FIG. 2, it was confirmed that the frictional force of the sample surface after the coating exhibited a significantly lower value than the frictional force of the sample surface before the coating. This means that the surface smoothness of the substrate after the coating was significantly improved.

### Example 5: Antithrombotic Activity

1. Platelet Adhesion Test

A platelet adhesion test is the most widely used in-vitro test using an SEM to observe a sample surface to check whether blood platelets are attached to a sample surface. First, platelet-rich plasma (PRP) was prepared. Whole blood was centrifuged at 1000 rpm for 10 minutes. When whole blood was centrifuged twice, the obtained plasma volume was about 10% of the whole blood volume. Then, a coated sample was immersed in the prepared PRP at 37° C. for 2 to 10 hours while shaking. This is a step of applying platelets to a sample to check the presence or absence of platelets attached to the surface of the coated sample. Next, a washing step was performed to wash out the remaining platelets unattached to the surface of the coated sample. The sample was washed with 0.1M phosphate buffered saline (PBS) having a pH of 7.4 and a temperature of 37° C. for minutes while shaking. A platelet fixation step was performed. The platelets attached to the surface of the sample were fixed by immersing the sample in 2% or 2.5% of a glutaraldehyde solution for 2 hours. Then, in a dehydration step, the samples were dehydrated stepwise with 50%, 75%, 90%, and 100% of aqueous ethanol solutions (EtOH:DW=v:v) for respective dehydration stages each of which was performed at 4° C. for 10 minutes. Next, the dehydrated sample was freeze-dried for 6 hours, sputtered with gold, and then subjected to measurement using an SEM.

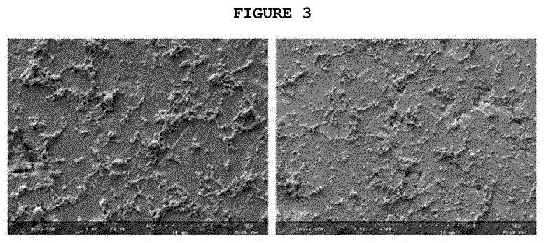

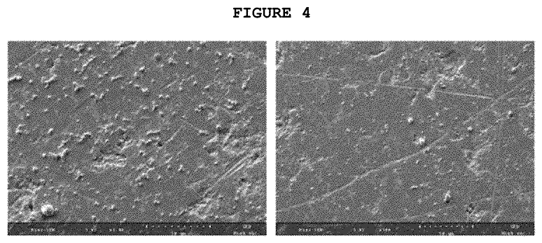

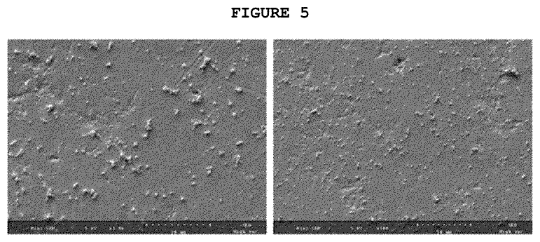

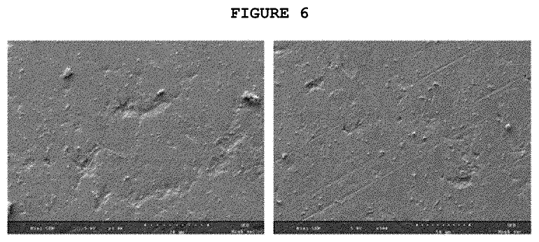

FIG. 3 shows the platelet adhesion test result of a control group (Co—Cr alloy-coated disk), and FIGS. 4, 5 and 6 show the platelet adhesion TEM test results of a heparin-coated substrate, a fucoidan-coated substrate, and an MPC-coated substrate, respectively. It was found that the platelet adhesion to the heparin-coated substrate, the fucoidan-coated substrate, and the MPC-coated substrate was significantly lower than the platelet adhesion to the control group.

2. Visual Observation Test

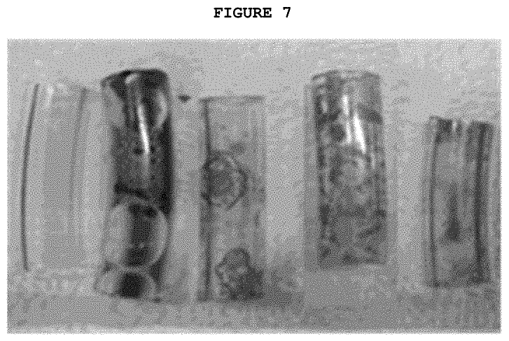

A visual observation test was also performed to test the performance of the antithrombotic coating composition of the present invention. Blood was circulated through a coated tube for 30 minutes and then the tube was washed with water. In this test, it was visually checked whether the blood remained on a coated region and an uncoated region after the tube was washed. Referring to FIG. 7, the control group (the second from the left) which is an uncoated sample shows a relatively large amount of blood remaining even after the sample was washed with water compared to its previous state (leftmost, meaning the state before the beginning of the test). However, the heparin-coated substrate (center), the fucoidan-coated substrate (the second from the right), and the MPC-coated substrate (rightmost) show that the adhesion of platelets thereto is significantly lowered after they were washed.

What is claimed is:

1. An antithrombotic coating composition comprising:
a first coating solution comprising at least one selected from the group consisting of an acrylic compound and a polyurethane compound;
a second coating solution comprising a polysaccharide and an antithrombotic substance; and
a cross-linking agent.

2. The antithrombotic coating composition of claim 1, wherein the antithrombotic substance is at least one selected from the group consisting of heparin, hirudin, H-heparin, HSI-heparin, streptokinase, neurokinase, fucoidan, and 2-methacryloyloxyethyl phosphorylcholine (MPC).

3. The antithrombotic coating composition of claim 1, wherein the first coating solution is to be bound to a substrate, and the second coating solution is to be bound, by the cross-linking agent, to the first coating solution bound to the substrate.

4. The antithrombotic coating composition of claim 1, wherein the acrylic compound is an acrylate polymer.

5. The antithrombotic coating composition of claim 1, wherein the polyurethane compound is polyether polyurethane.

6. The antithrombotic coating composition of claim 1, wherein the polysaccharide is hyaluronic acid.

7. The antithrombotic coating composition of claim 1, wherein the cross-linking agent is a polyaziridine-based or polyisocyanate-based cross-linking agent.

8. The antithrombotic coating composition of claim 1, wherein the second coating solution further comprises polyether polyurethane.

9. The antithrombotic coating composition of claim 8, wherein the polysaccharide and the polyether polyurethane are present in a weight ratio of 10:0.5 to 2.

10. An antithrombotic coating method comprising:
a) coating a surface of a substrate with a first coating solution containing at least one selected from the group consisting of an acrylic compound and a polyurethane compound;
b) drying the coated substrate resulting from step a);
c) coating the dried substrate resulting from step b) with a second coating solution containing a polysaccharide and an antithrombotic substance; and
d) drying the coated substrate resulting from step c).

11. The antithrombotic coating method of claim 10, wherein the antithrombotic substance is at least one selected from the group consisting of heparin, hirudin, H-heparin, HSI-heparin, streptokinase, neurokinase, fucoidan, and 2-methacryloyloxyethyl phosphorylcholine (MPC).

12. A antithrombotic substrate coated by the method of claim 10.
